(19) United States
(12) Patent Application Publication
(10) Pub. No.: US 2004/0096354 A1
(43) Pub. Date: May 20, 2004

(54) Ozone deodorizing and sterilizing method and device

(76) Inventors: Shinnosuke Nomura, Osaka (JP); Naoya Kitamura, Kyoto (JP)

Correspondence Address: WENDEROTH, LIND & PONACK, L.L.P., 2033 K STREET N. W., SUITE 800, WASHINGTON, DC, 20006-1021, US

(21) Appl. No.: 10/298,066

(22) Filed: Nov. 18, 2002

(51) Int. Cl. A61L 9/00; A61L 2/00

(52) U.S. Cl. 422/23; 422/22; 422/28; 422/186.07

(57) ABSTRACT

An ozone deodorizing/sterilizing device is proposed which includes an air pump for supplying pressurized air, an ozone generator for producing ozone by silent discharge, and an ozone spray device having a spray nozzle for atomizing water with the pressurized air containing ozone as a driving fluid and spraying it with air. The spray nozzle is formed to atomize water by setting the nozzle hole diameter so that water will be atomized into ultrafine volatile fog drips and fog drips will be sprayed within a predetermined range from the spray nozzle and the target space to be sterilized will be kept from getting wet.

### BACKGROUND OF THE INVENTION

This invention relates to an ozone deodorizing and sterilizing method and device for deodorizing and sterilizing by ozone by atomizing water with air containing ozone as a driving fluid and mixing the ozone-containing air and volatile mist drips and spraying the mixture.

Ozone has a strong oxidizing power, and therefore it is widely used in many fields such as deodorization, sterilization, insecticide, sterilization, decoloration, freshness retainment and environmental improvement. Its typical use includes applications to purification of tap water, sewage treatment, deodorization in foul odor environments, food processing, alga elimination, fisheries, stock farming, agriculture, etc. When ozone is used in such various kinds of applications, well-known methods include washing with water in which is dissolved ozone, spraying ozone water, and sprinkling air containing ozone directly on target objects.

Besides these general methods, a method is known in which spray drips containing ozone are sprayed or sprinkled. In this method, pressurized gas containing ozone is fed to a spray nozzle in which water is sucked and mixed with a suction function (or ejector effect) by the pressurized air, and it is sprayed through the nozzle hole in the form of ultrafine mist drips for deodorization and sterilization. Compared with the method in which ozone water is sprayed or the method in which ozone is directly sprinkled, this method has advantages that ozone can be effectively contained in mist drips and can be sprinkled and that safety is higher.

An ozone spray device in which two fluids are mixed in a nozzle and sprayed is described in JP patent publication 2000-316956 proposed by the present inventor. It includes a spray nozzle provided with a passage through which air containing ozone flows and a passage through which a liquid from a liquid supply source flows, so as to be independently of and parallel to each other, with the ends of the respective passages provided concentrically with each other at the discharge port so that the concentric flow-out ends will form an ejector for sucking liquid with the energy of air, and a container for storing liquid.

With the spray nozzle of the prior art ozone spray device, a passage for sucking and passing liquid parallel to the passage for gas is provided offset from the central position so as not to intersect the passage for gas, which passes the central position. The passage for liquid has its portion for communication with the atmosphere provided so as to extend vertically through the nozzle body. The end of the nozzle body is formed in a protruding shape, the end of the passage for gas is provided as a protruding end with a conical gap formed between the nozzle body and a nozzle cap connected to the nozzle body such that this protruding end will be located slightly inner than the discharge port of the cap.

In this publication, a small portable ozone sterilizing device provided with the above ozone spray device is also described. In the ozone sterilizing device, air containing ozone is fed from an ozone generator for producing ozone by silent discharge from air supplied from an air pump for compressing air, and the air is fed to the spray nozzle of the ozone spray device and the air containing ozone is mixed with water to spray it as sprayed droplets.

There is a report in which only ozone is sprinkled in the air in rooms such as hotel rooms to investigate sterilizing/deodorizing effects by ozone. According to the report, the lower the humidity, or the lower the sprinkled amount of ozone, the lower the effects. In particular, as for the humidity in the air, unless it is at least 60% or over, the sterilizing/deodorizing effects are low. Thus, if sterilization/deodorization is carried out by sprinkling only ozone, as is stipulated in the Labor Safety Hygiene Law, a method is general in which such an amount of ozone is sprinkled that the concentration of ozone contained in the air reaches a level of 0.1 ppm or less and this treatment is repeated every day to gradually increase the effect. But this method is too time-consuming and is not suitable for e.g. hotel rooms where guests using them change every day.

On the other hand, a method is conceivable in which ozone water containing ozone dissolved in water is sprayed through a spray nozzle to sprinkle it in a room. In this method, by setting the pressure at which the ozone water is fed into the spray nozzle to a relatively high pressure of e.g. 3-5 kg/cm², it is possible to spray mist drips sprayed by the nozzle in an ultrafine state with their diameter equal to or less than a predetermined diameter. Thus, the mist drips vaporize after having been sprayed through a nozzle. But since it is impossible to dissolve in the ozone water a large amount of ozone over the dissolving rate which is determined by the temperature of the ozone water, the amount of ozone contained in such ozone water is so small that one cannot expect sterilizing/deodorizing effects by ozone even if such ozone water is sprayed.

Therefore, instead of the conventional two sterilizing/deodorizing methods, it is conceivable to apply the ozone sterilizing method by the above publication to sterilization/deodorization in rooms such as hotel rooms. While the small portable ozone sterilizing device disclosed in the publication is called a sterilizing device, it has a strong effect and can be used not only for sterilization but for various applications such as sterilization, deodorization and freshness retainment. For example, it is possible to carry out sterilization, deodorization and freshness retainment for kitchen facilities for home or business use and perishable foods handled therein. Since one person can freely move the entire device, it is extremely convenient. But this device cannot be used in hotel rooms and hospital rooms as it is.

This is because in hotel and hospital rooms, there are many equipments that have to be kept from getting wet such as furniture like beds, illuminating equipments and floor mats. If sprayed droplets are sprinkled for over a predetermined time period, they will turn to waterdrops, and the waterdrops collect to form a wet state. If these equipments get wet, various germs, airborne germs and molds tend to stick to water. This is hygienically unfavorable. And this is because the the diameter of the sprayed droplets from the spray nozzle is too large. While they are airborne, the droplets having a larger-than-predetermined-value diameter do not vaporize completely, but settle by gravity, remain waterdrops.

Thus, for mist drips sprayed through the spray nozzle of the ozone spray device of the ozone sterilizing device, the present inventors have conceived a method and a device which can produce fog drips that can vaporize (dry fog) by measuring the limit of the fog drip diameter within which the fog drips vaporize, finding the conditions under which the fog drips will not settle by gravity to form waterdrops, and setting the spray nozzle diameter adapted thereto and the conditions of the pressure of air containing ozone.

An object of the present invention is to provide an ozone deodorizing/sterilizing method and device which provides deodorizing/sterilizing effects with high efficiency without getting the floor wet.

### SUMMARY OF THE INVENTION

According to the present invention, there is provided an ozone deodorizing/sterilizing method comprising the steps of feeding air pressurized to a predetermined pressure into a high-voltage discharge space, producing pressurized air containing ozone produced from oxygen in the air by silent discharge in the discharge space, feeding the pressurized air to a spray nozzle where the pressurized air is mixed with water, atomizing water with the pressurized air as a driving fluid by applying nozzle outlet resistance to the pressurized air to form ultrafine volatile fog drips to such an extent that water will vaporize, and spraying it to carry out deodorizing/sterilizing treatment in a fog drip mixing zone or in a high-humidity atmosphere.

According to the present invention, there is also provided an ozone deodorizing/sterilizing device comprising an air pressurizing means for pressurizing air to a predetermined pressure, an ozone generator for producing ozone in the pressurized air by silent discharge by applying high voltage between electrodes between which is mounted a dielectric, and an ozone spray device having a spray nozzle for atomizing water with the pressurized air containing ozone as a driving fluid and spraying it with air, the spray nozzle being formed to atomize water by setting the nozzle hole diameter so that water will be atomized into ultrafine volatile fog drips, whereby deodorizing/sterilizing treatment can be carried out with ozone-containing air in a fog drip mixing zone or in a high-humidity atmosphere.

According to the ozone deodorizing/sterilizing method and device of this invention, it is possible to achieve deodorizing/sterilizing effects with high efficiency without wetting floor surfaces with water in rooms and spaces of which users change almost everyday. This is because in deodorizing and sterilizing by sucking water with pressurized air containing ozone, spraying it to sprinkle fog drips on the target object or space, it is sprayed as ultrafine fog drips of such a size that while the fog drips are airborne, they will vaporize. The limit of ultrafine fog drips that can vaporize is 10 μm, and the particle diameter has to be equal to or less than 10 μm.

In order to sterilize with air containing ozone by spraying fog drips with ozone-containing pressurized air with the ozone sterilizing device disclosed in the above-described patent publication, the entire system is set such that the amount of ozone generated will be a maximum amount with maximum efficiency with a single ozone generator. Based on the principle that ozone is generated by subjecting air flow to silent discharge, the pressure and the flow rate of the pressurized air are set as low as possible. For example, the pressure is set at a low pressure of 0.03 MPa [0.3 kgf/cm^{2].}

Since fog drips sprayed through a spray nozzle with the pressure set at such a low pressure are all large in size and the particle diameter exceeds 10 μm, when sprayed and airborne, they will drop onto the floor by gravity in a short time, so that they produces a wet state on the floor. In contrast, according to this invention, in order to spray them so that the particle diameter of fog drips sprayed are equal to or smaller than the above predetermined particle diameter, the discharge pressure at the spray nozzle is set at such a high pressure that they can become ultrafine volatile fog drips.

In order to set the pressure of pressurized air at the spray nozzle at such a high pressure, the pressure of pressurized air is increased by applying resistance to the nozzle hole outlet by forming the nozzle hole diameter as small as possible. This pressure is such a pressure that the fog drips will become volatile fog drips. For example, it is about 0.07-0.08 MPa. At such a high pressure, the ozone generating efficiency markedly drops, so that it is impossible to obtain a necessary ozone amount with a single generator. But the necessary ozone amount has to be obtained even if the ozone generating efficiency is sacrified.

The necessary ozone amount is set in view of safety of workers who sprinkle ozone, at such an amount that the ozone concentration in the atmosphere in which ozone has been sprinkled ensures safety. Also, it is necessary to set the flow rate of the pressurized air when sucking water with pressurized air containing ozone at the spray nozzle and spraying it. This is necessary to set the distance range to which the fog drips sprayed from the spray nozzle reach in the form of fog drips. Thus, the flow rate of the pressurized air is set such that the fog drip mixed area in which fog drips sprayed are mixed are in a predetermined range, e.g. about 2 meters.

Other features and objects of the present invention will become apparent from the following description made with reference to the accompanying drawings, in which:

### BRIEF DESCRIPTION OF THE DRAWINGS

FIG. 1 is a schematic view of the device embodying the present invention;

FIG. 2A is a front view of the same;

FIG. 2B is a side view of the same;

FIG. 3 is a vertical sectional view of the ozone spray device;

FIG. 4A is a sectional view of the ozone generator;

FIG. 4B is an enlarged view of the spray nozzle;

FIG. 5A is a sectional view taken along line Va-Va of FIG. 4A;

FIG. 5B is a sectional view taken along line Vb-Vb of FIG. 4A; and

FIG. 6 is a view showing how the device is used.

### DETAILED DESCRIPTION OF THE PREFERRED EMBODIMENT

Hereinbelow, the embodiment of this invention will be described with reference to the drawings.

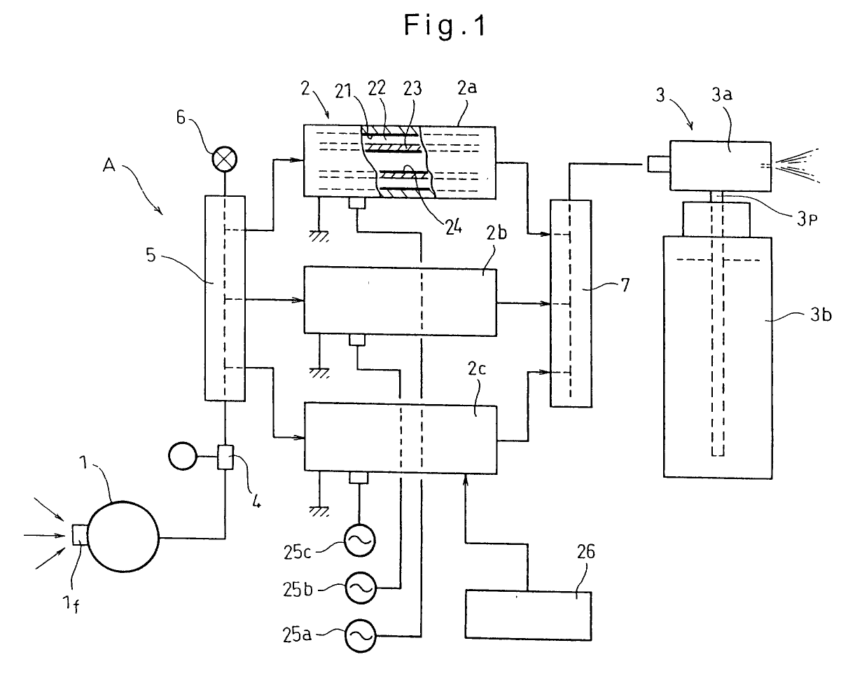

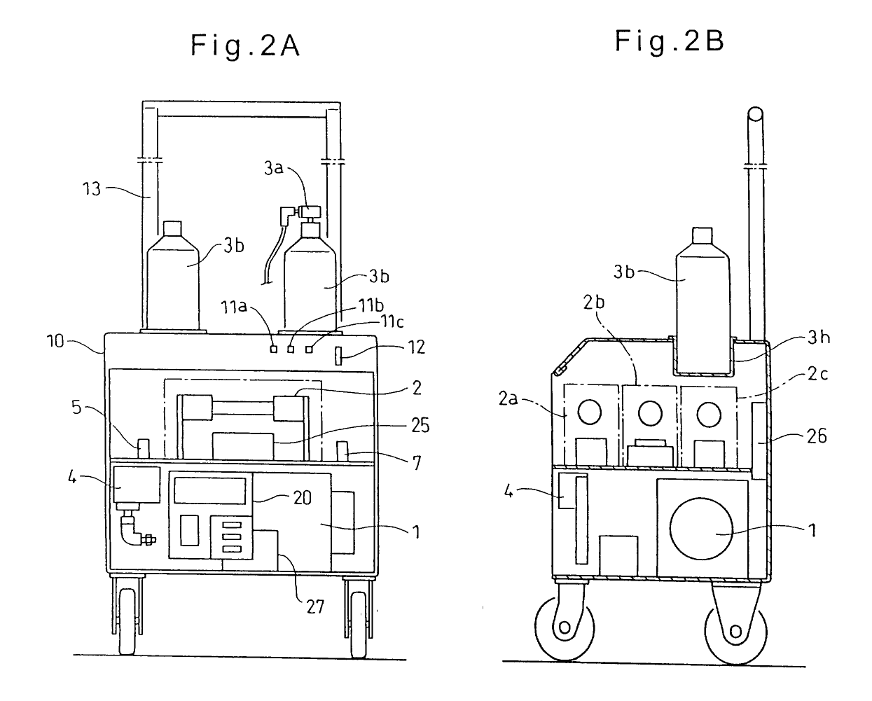

FIG. 1 is a schematic view showing the entire structure of the ozone deodorizing/sterilizing device embodying the present invention. FIGS. 2A, 2B are views showing the structure of this device. As shown, the ozone deodorant/sterilizing device A includes an air pump 1 for pressurizing air to a predetermined pressure, ozone generators 2 for producing ozone from the pressurized air by silent discharge, and an ozone spray device 3 for atomizing water in a spray nozzle 3a with ozone-containing pressurized air as a driving fluid and spraying it together with the air.

The air pump 1 sucks air through a filter If and pressurizes it. The pressurized air is distributed in parallel to a plurality of (three in the embodiment) ozone generators 2 (2a, 2b, 2c) by a distributing header 5 through a shutoff valve 4 having a pressure switch. An air adjusting valve 6 is provided. The ozone generators 2 (2a, 2b, 2c) are of commercially available silent discharge type, and are provided with a cylindrical glass dielectric 23 in a cylindrical grounded electrode 21 (cathode) with a gap 22 therebetween. In the center, a columnar high-voltage electrode 24 is provided in contact with the dielectric 23. Due to silent discharge by high voltage applied between the high-voltage electrode 24 and the pressurized air flowing through the gap 22, part of the oxygen (O₂) in the air is dissociated to produce ozone (O₃).

The ozone generators 2 (2a, 2b, 2c) are connected to independently provided high-frequency, high-voltage power sources 25 (25a, 25b, 25c) to supply high voltage. High-frequency high voltage is supplied to the high-voltage electrodes 24. In the illustrated embodiment, high voltage of 17000 V at high frequency of 20000 Hz (power: 14 W) is supplied by the high-voltage power sources 25.

The ozone generators 2 (2a, 2b, 2c) have a length of 200 mm and an outer diameter of 19.5 mm. The pressurized air is supplied by the air pump 1 at 0.8-1.2 kgf/cm². But the pressure of pressurized air in the ozone generators 2 (2a, 2b, 2c) is adjusted by an air adjusting valve 6 to 0.7-0.8 kgf/cm² [0.07-0.08 MPa]. A cooling fan 26 is provided to cool heat buildup due to discharge equilibrium in the ozone generators 2 (2a, 2b, 2c). The pressurized air containing ozone produced in the plurality of ozone generators 2 is collected into a collecting header 7, and the whole flow of pressurized air is fed to the spray nozzle 3a. A supply pipe 3p for supplying water is connected to the spray nozzle 3a so that water can be supplied from a container 3b storing water through the supply pipe 3p. Its details will be described later.

FIGS. 2A and 2B show an outline of the internal arrangement in which the members of the ozone deodorizing/sterilizing device are mounted in a housing 10. At the bottom of the housing 10, caster wheels are provided. Thus the device is designed portable so that it can be moved by pushing it manually. In the housing 10, a plurality of ozone generators 2 (2a, 2b, 2c), high-voltage power sources 25 (25a, 25b, 25c), fan 26, distributing header 5, and collecting header 7 are arranged on the upper level, while the air pump 1, shutoff valve 4 with a pressure switch, relays and terminal boards, a circuit board 20 including a control circuit, a transformer 27 for power voltage conversion, etc. are arranged on the lower level. Air piping and electric wiring are omitted because they complicate the figure.

On the front upper portion of the housing 10, display lamps 11a, 11b, 11c that indicate the states of the ozone generators 2 (2a, 2b, 2c), and an alarm lamp 12 for warning that the pressure switch is activated are provided. In a top plate of the housing 10, two shallow recesses 3h for receiving the container 3b of the ozone spray device 3 are formed. For piping for supplying ozone-containing air to the spray nozzle 3a, a flexible ozone tolerant pipe, for example a fluoro material, is used. The length of the flexible ozone tolerant pipe is determined so that during use of the ozone spray the container 3b and the spray nozzle 3a taken out of the recess 3h can be moved in a predetermined distance range while spraying on target objects. As a hanger for hooking the vinyl pipe when the ozone spray device 3 is not used, a U-shaped frame 13 is mounted on top of the housing 10.

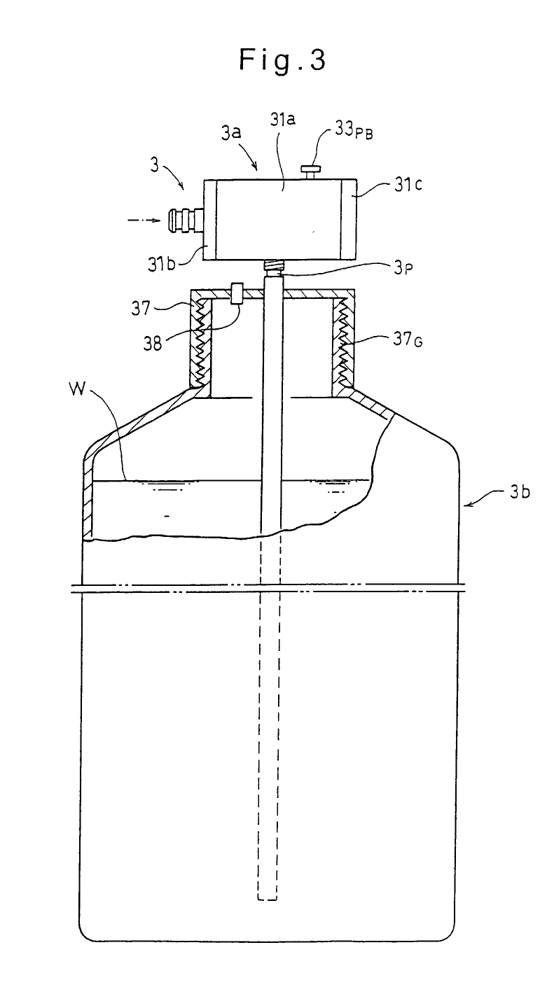

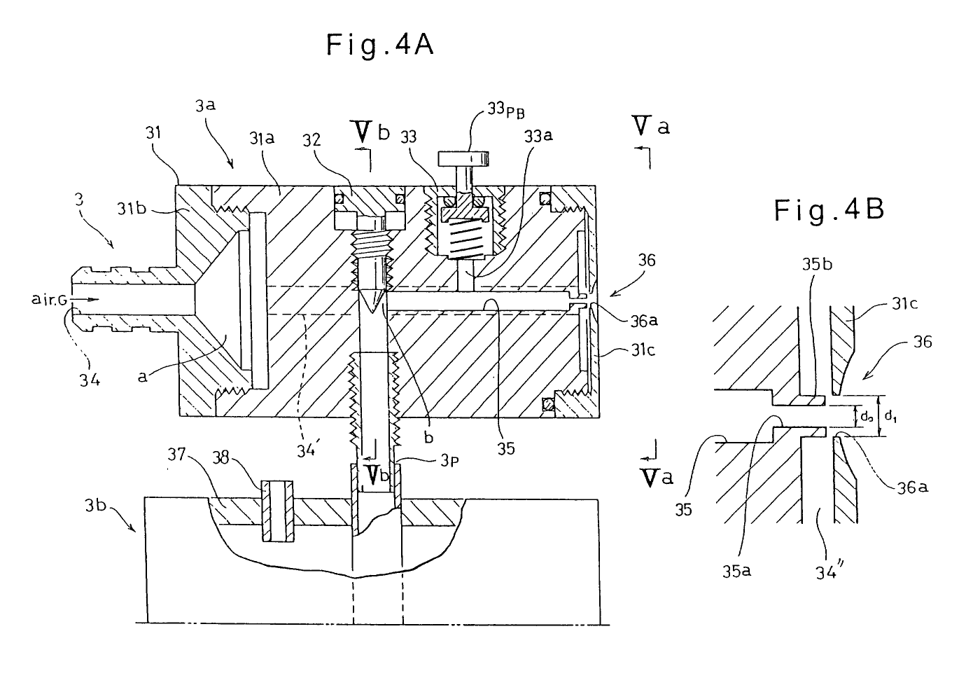

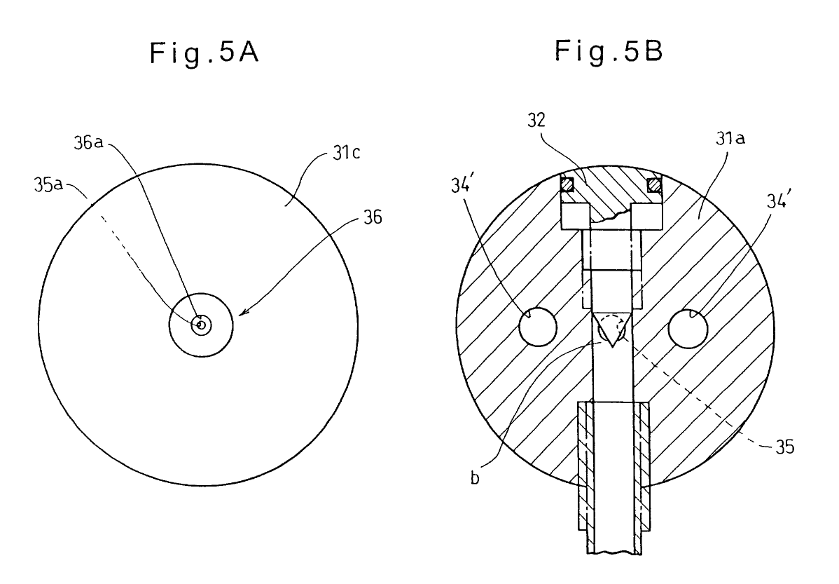

FIG. 3 is a longitudinal sectional view of one embodiment of the ozone spray device. The spray nozzle 3a of the ozone spray device 3 is connected to the container 3b through a connecting pipe 3p, and is structured to suck water W stored in the container 3b from the container 3b with ozone-containing air G that is supplied to the spray nozzle 3a as a driving fluid, and spray it through a discharge port 36 (FIG. 4A) as fog drips. The spray nozzle 3a includes a nozzle body 31a, a connecting portion 31b threadedly connected to one end thereof, and a cap 31c forming the discharge port 36.

The connecting portion 31b is formed in the shape of a circular plug and has a connecting pipe protruding from the end face thereof for receiving ozone-containing air G. A passage 34 defined by the inner periphery of the connecting pipe has its diameter conically expanded at a position a in the connecting portion 31b. The connecting portion 31b is threadedly engaged in a connecting hole formed in one end of the nozzle body 31a. To the connecting hole, ends of two passages 34′ formed in the nozzle body 31a open and communicate with the passage 34 to divide ozone-containing air G into the two passages 34′.

The nozzle body 31a is in the form of a circular column (FIG. 4). At its center, a passage 35 for axially feeding water is formed so as to be slightly longer than half the length of the nozzle body. The inner end of the passage 35 is bent in the shape of L so as to communicate with the top end of the connecting pipe 3p. A needle valve 32 and a stop valve 33 for stopping the flow of water are provided radially. The former extends toward a corner portion b of the passage 35. The needle valve 32 is provided as a flow control valve for suitably setting the amount of water flowing through the passage 35 such that the particle diameter of the below-described fog drips will be of an optimum size.

The stop valve 33 has its valve body threadedly engaged in a recess for receiving the valve body, and a small hole 33a at an end of the recess communicates with the passage 35. The valve body has a spring, a valve rod and a valve seat ring housed in a valve cup. Normally, the valve rod is pushed up by the spring to close a hole for the valve rod with the valve seat ring. When a button 33PB at the top end of the valve rod is pushed down, the interior of the recess communicates with the outer air, so that the interior of the passage 35 is sucked, thus shutting off the flow of water flowing in the passage 35.

The passage 35 communicates at the tip side of the nozzle body 31a with a nozzle hole 35a (with a diameter d0) having a reduced diameter. The nozzle hole 35a has a discharge port of the same diameter in a protruding portion 35b protruding into a shallow recess 34″ formed in the front of the nozzle body 31a (FIG. 4B). The above-said two passages 34′ are formed through the nozzle body 31a so as to be parallel to the passage 35 with their other ends opening at the front side of the nozzle body 31a into the recess 34″.

At the tip end of the nozzle body 31a, the cap 31c is threadedly engaged and mounted. In the center of the cap 31c, a discharge port 36a (of a diameter d1) is formed. Their dimensions are set so that a predetermined gap remains between the cap 31c and the tip of the protruding portion 35b. Thus, when ozone-containing air G flowing from the passage 34 through the passages 34′ into the recess 34″ passes through the predetermined gap to the discharge port 36a, it sucks water in the passage 35 due to negative pressure applied to the discharge port of the protruding portion 35b, which opens to the same discharge port 36a. Thus, water coming out the outlet port of the protruding portion 35b collides hard against the air G as the driving fluid, and is discharged in the form of ultrafine fog drips.

The illustrated spray nozzle 3a (FIG. 4B) is set such that the diameter d0 of the nozzle hole 35a is 0.7 mm, diameter d1 of the discharge port 36a is 1.1 mm, discharge pressure p of the pressurized air G immediately coming out of the discharge port 36a is 0.8 kgf/cm² [0.08 MPa], flow rate Q of the pressurized air G is 12 liters/minute, and the flow rate of sprayed water is 10 cc/minute. In particular, the reason why the diameter d0 of the nozzle hole 35a is set to 0.7 mm is because it is the minimum diameter workable by a mechanical working means like a boring drill, with a minimum working cost and further in view of the required amount of fog drips (if the diameter is too small, it is impossible to ensure the required amount of fog drips).

The connecting pipe 3p connected to the spray nozzle 3a is fixed to a cap 37 that is in threaded engagement with the top end of the container 3b at a threaded portion 37G. A communicating pipe 38 communicates with the atmosphere. When water in the container 3b has been used up, the spray nozzle 3a is fitted on another container 3b which stores water beforehand. At this time, spray work can be continued by removing the cap 37 together with the connecting pipe 3p by disengaging the threaded portion 37G, and bringing it into threaded engagement with the other container 3b.

The ozone deodorizing/sterilizing device A embodying the present invention, which has the structure as described above, is extremely effective in target objects or spaces (e.g. hotel rooms) where quick deodorizing/sterilizing treatment is required but getting wet is severely restricted. When starting ozone deodorizing/sterilizing work, a start switch (not shown) mounted on the housing 10 of the device A is turned on to start the air pump 1 and the high-voltage power source 25 of the ozone generators 2, and the ozone spray device 3, which has the spray nozzle 3a and the container 3b, is removed from the housing 10.

Some time after turning on the start switch, pressurized air G supplied to the spray nozzle 3a will become ozone-containing air. After a predetermined time has passed, water will be sucked through the spray nozzle 3a by the pressurized air G and fog drips will be sprayed. By directing the spray nozzle tip at the target object or space, it is possible to carry out deodorizing/sterilizing work. When it is desired to temporarily stop spraying work, a push button 33PB is pushed in.

Fog drips atomized from the spray nozzle 3a together with ozone-containing air as the driving fluid are in the form of ultrafine fog drips of such a size that they are still visually recognizable as airborne mist in the distance range of about 2 meters in the spraying direction, but at distance out of this range, they will vaporize and cannot be seen. Thus, ultrafine fog drips that can be vaporize have to be 10 μm or less in diameter.

It has been confirmed by experiments that fog drips having a diameter of 10 μm or over may partially vaporize but will never vaporize completely, so that in a predetermined distance range in the spraying direction, they will drop on the floor in the form of waterdrops and the floor become wet in a short time (about several minutes). Such coarse fog drips cannot be sprayed on target objects or space of this device. Thus, in order to spray ultrafine volatile fog drips from the spray nozzle 3a, the discharge pressure of ozone-containing air G used as the driving fluid in the spray nozzle 3a is preferably as high as possible.

But since the ozone-containing air G supplied to the spray nozzle 3a as the driving fluid is directly supplied from the ozone generators 2 (2a, 2b, 2c) through the collecting header 7, the pressure set at the spray nozzle 3a is substantially equal to the pressure in the ozone generators 2 (if the pressure loss in piping is ignored), so that the condition has to be met that the pressure in the ozone generators 2 has to be substantially equal to the discharge pressure at the spray nozzle. But in silent discharge type ozone generators 2, as the air pressure increases, the ozone generating efficiency decreases, so that it is impossible to obtain a predetermined amount of ozone generated.

The results of measurement in which the amount of ozone was measured while changing the discharge pressure at the spray nozzle 3a for the plurality of ozone generators 2 (2a, 2b, 2c) are shown below.

TABLE 1

| Discharge | Ozone | Amount |
|---|---|---|
| pressure | concentration | of ozone |
| 0.04 Mpa | 700 ppm | 1080 mg/h (18 mg/min) |
| 0.05 Mpa | 630 ppm | 972 mg/h |
| 0.06 Mpa | 495 ppm | 763 mg/h |
| 0.07 Mpa | 410 ppm | 632 mg/h |
| (0.8 kgf/cm²) 0.08 Mpa | 350 ppm | 540 mg/h (9 mg/min) |
| 0.09 Mpa | 294 ppm | 453 mg/h |

The ozone concentrations and ozone amounts are the total for three ozone generators 2. The discharge flow rate of air from the spray nozzle 3a during measurement was 12 liter/min at pressure of 0.08 MPa. When the pressure was reduced to 0.04 MPa, it decreased to about 11 liter/min but did not change so much.

As is apparent from the measurement results, when the discharge pressure is reduced, the amount of ozone contained in the pressurized air increases. When the pressure decreases from 0.08 MPa to 0.04 MPa, the amount of ozone doubles. But when the discharge pressure decreases to 0.04 MPa, the particle diameter of the fog drips atomized from the spray nozzle 3a would exceed 10 μm, so that the condition for fog drip size for vaporization will not be met. Thus, the discharge pressure which meets the condition of fog drips that they completely vaporize was determined. As a result, it was confirmed that the discharge pressure has to be at least 0.07-0.08 MPa or over. Thus, for this device, the discharge pressure is set at 0.08 MPa.

For reference, in the conventional ozone spray device described in the aforementioned patent publication, the discharge pressure at the spray nozzle end was set at a low pressure of 0.03 MPa [0.3 kgf/cm²] so that the ozone generating efficiency in the ozone generators would be high. If it is possible to set at such a low pressure, it will be possible to keep the ozone generating efficiency high. But the condition of fog drips for complete vaporization will not be met. Thus, fog drips having a particle diameter of 10 μm or over are sprayed without being completely vaporized.

In contrast, in the device of the above embodiment, the discharge pressure from the spray nozzle 3a is set to meet the condition that fog drips will completely vaporize, and the reduction in the amount of ozone is compensated by reducing the flow rate of pressurized air in one ozone generator 2, thereby supplying a required amount of ozone-containing pressurized air to the spray nozzle 3a. Thus, in this embodiment, a plurality of ozone generators 2 (2a, 2b, 2c) are provided with reduction in the flow rate in each of the ozone generators 2a, 2b, 2c. In the embodiment, by use of three ozone generators 2a, 2b, 2c, pressurized air G containing a required amount of ozone for deodorizing/sterilizing treatment is obtained.

The required minimum amount of ozone is set as follows. As described above, ozone has deodorizing/sterilizing effects and the greater the amount of ozone, the higher the effects. But on the other hand, ozone has properties that if it is too large in amount, it is dangerous to human bodies. Naturally, safety of operators who carry out deodorizing/sterilizing treatment has to be taken into consideration. When fog drips are sprinkled from the spray nozzle 3a together with ozone-containing air for a certain time period, ozone disperses and returns to the operator who is behind the spraying point. Thus, the operator will inhale ozone-containing air. If the ozone concentration in the ozone-containing air is high, a dangerous situation may occur to the operator.

Thus, for operators working in such work environments, the Labor Safety Hygiene Law stipulates that the ozone concentration must not exceed 0.1 ppm per unit volume of air (1 liter). Thus, when ozone is sprinkled from the spray nozzle of the embodiment, in view of higher safety, the target is set so that the concentration of ozone in the air inhaled by the operator when sprinkled for 3-5 minutes should be 0.05 ppm or less. As the required minimum amount of ozone under such conditions, the amount to be generated in the ozone generators 2 is set to 350 ppm at the discharge pressure of 0.08 MPa.

The amount of ozone is the total of the amounts obtained by three ozone generators 2a, 2b, 2c. Thus, if an ozone generator which can generate the same ozone amount is available, one such ozone generator will suffice. But if only one of the ozone generators 2 of the illustrated size is used, it would be impossible to obtain the same ozone amount and the flow rate of air fed into this single ozone generator will triple, so that due to reduced efficiency resulting from increased flow rate, only an ozone amount smaller than ⅓ of the above ozone total amount could be obtained.

Thus, in order to obtain the same ozone amount as the above total amount with only one ozone generator, the length of the ozone generator has to be at least tripled, or the diameter has to be increased to √3-fold, and further according to the increased length or diameter, the high-voltage electrodes and high-voltage power sources 25 have to be changed to one of such a size that a 3-fold amount of ozone can be obtained. In the illustrated embodiment, since ozone generators 2 designed for use with commercial high-voltage power sources of a predetermined standard, by using three such ozone generators 2, a required minimum amount of ozone is ensured. Thus, a larger number of ozone generators 2 than in the embodiment, e.g. four or five of them may be used, but this is not preferable because the cost increases.

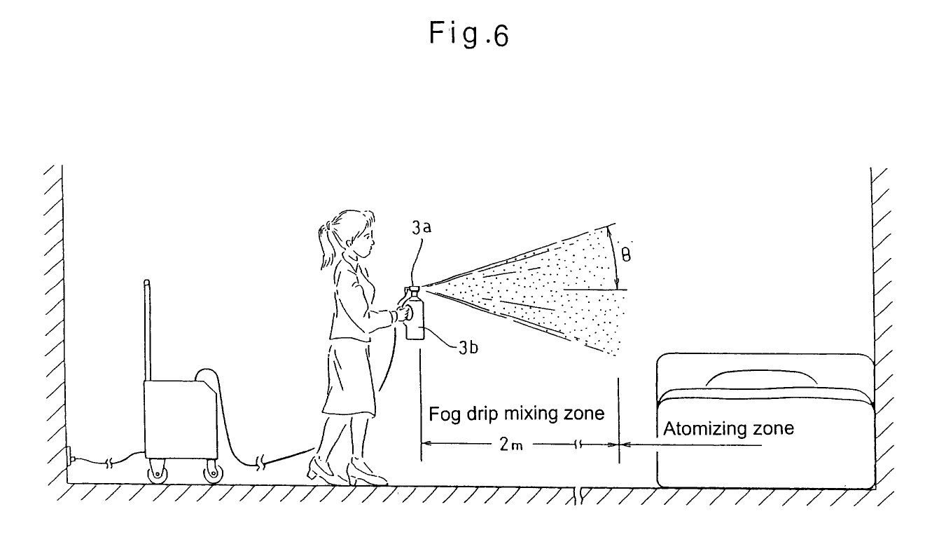

Also, the setting of the number of ozone generators is related to the diameters of the air nozzle 35a and the fog drip discharge port 36a of the spray nozzle 3a. In particular, the diameter of the nozzle 35a is set to the minimum diameter (0.7 mm) obtained by machining so that the air discharge pressure can be set high (0.07-0.08 MPa), and the diameter of fog drips will be small. Further, as the spray distance of fog drips sprayed from the spray nozzle 3a, as shown in FIG. 6, it is necessary to supply a predetermined air discharge amount required to spray so that a fog drip mixing zone of about 2 meters will be produced.

Thus, if an air pump 1 having a capacity corresponding to this spraying distance is used, the flow rate of pressurized air would be too large for the single ozone generator 2, so that in the ozone generator 2, both the flow rate and pressure will exceed the rated values. This lowers efficiency. Thus, a required amount of ozone is obtained with a plurality of (three) of them by reducing the flow rate to the rated state. Volatile fog drips sprayed from the spray nozzle 3a thus set will have, as shown in FIG. 6, a radiating angle θ of about 30° (at one side) an the fog drip mixing zone.

When fog drips are sprayed from the device A of this embodiment in which the size, capacity, number of units are determined as above for the spray nozzle 3a, air pump 1 and ozone generators 2, the spray distance of about 2 meters from the spray nozzle 3a becomes the fog drip mixing zone. Even though fog drips are ultrafine particulate, their misty state can be visually recognizable in the zone. But outside this zone, they can no longer be seen but will vaporize. While they are not seen in the vaporized zone, ozone smell is slightly recognizable in this zone, so that it is confirmed from actual experience that fog drips have vaporized.

When carrying out deodorizing/sterilizing treatment with fog drips from the spray nozzle 3a, it is done such that deodorizing/sterilizing effects will be applied to the target object or space in the fog drip mixing zone whenever possible. This is because it is known that the deodorizing/sterilizing effect by ozone in the air of high humidity of 60% or over is far higher in effect than in the air of lower humidity. While the deodorizing/sterilizing effect by ozone in the vaporized zone in which fog drips have vaporized, is naturally high, the deodorizing/sterilizing effect in the droplet atmosphere before vaporizing is further higher than in high-humidity air.

In the above embodiment, in order to obtain a required amount of ozone, a plurality of (three) ozone generators are used. They are conventional standard ozone generators and used to reduce the entire cost. Thus, it is of course possible to use only one ozone generator to obtain the required ozone amount. In this case, as described above, it is necessary to increase the length or diameter of the single ozone generator. If the high-voltage power sources are also correspondingly increased in size, this is feasible.

As described above in detail, in the ozone deodorizing/sterilizing method and device of this invention, pressurized air is fed into a high-voltage discharge space, water is sucked and sprayed in the spray nozzle with ozone containing pressurized air produced by silent discharge to carry out deodorizing/sterilizing treatment with ozone contained in the mixing zone of volatile fog drips or the high-humidity atmosphere in which the fog drips have vaporized obtained by setting the discharge pressure to a pressure at which volatile fog drips can be produced. Thus, even if the target object or space to be deodorized or sterilized has to be kept from getting wet, it is possible to carry out deodorizing/sterilizing treatment with ozone by spraying fog drips.

What is claimed is:

1. An ozone deodorizing/sterilizing method comprising the steps of feeding air pressurized to a predetermined pressure into a high-voltage discharge space, producing pressurized air containing ozone produced from oxygen in the air by silent discharge in said discharge space, feeding the pressurized air to a spray nozzle where the pressurized air is mixed with water, atomizing water with the pressurized air as a driving fluid by applying nozzle outlet resistance to the pressurized air to form ultrafine volatile fog drips to such an extent that water will vaporize, and spraying it to carry out deodorizing/sterilizing treatment in a fog drip mixing zone or in a high-humidity atmosphere.

2. An ozone deodorizing/sterilizing method as claimed in claim 1 wherein the discharge pressure for producing said ultrafine volatile fog drips is 0.07 MPa or over.

3. An ozone deodorizing/sterilizing method as claimed in claim 1 or 2 wherein the amount of ozone contained in said pressurized air is set such that the concentration of ozone in the atmosphere in which sprayed fog drips have vaporized is below a predetermined value in view of safety to human bodies.

4. An ozone deodorizing/sterilizing method as claimed in claim 1 wherein the flow rate of said pressurized air is set such that the fog drip mixing zone in which sprayed fog drips are mixed is in a predetermined distance from the spray point.

5. An ozone deodorizing/sterilizing device comprising an air pressurizing means for pressurizing air to a predetermined pressure, an ozone generator for producing ozone in the pressurized air by silent discharge by applying high voltage between electrodes between which is mounted a dielectric, and an ozone spray device having a spray nozzle for atomizing water with the pressurized air containing ozone as a driving fluid and spraying it with air, said spray nozzle being formed to atomize water by setting the nozzle hole diameter so that water will be atomized into ultrafine volatile fog drips, whereby deodorizing/sterilizing treatment can be carried out with ozone-containing air in a fog drip mixing zone or in a high-humidity atmosphere.

6. An ozone deodorizing/sterilizing device as claimed in claim 5 wherein the diameter of the hole of said spray nozzle is formed to 0.7 mm, which is the lower limit obtained by machining.

7. An ozone deodorizing/sterilizing device as claimed in claim 5 or 6 wherein said ozone generator is formed to have a length and a diameter which correspond to a capacity required to produce an amount of ozone contained in pressurized air such that the ozone concentration in the atmosphere in which sprayed fog drips have vaporized is below a predetermined value in view of safety to human body.

8. An ozone deodorizing/sterilizing device as claimed in claim 7 wherein a plurality of said ozone generators are provided so that the total amount of the ozone generated in said each ozone generator is a predetermined value.
